(19) United States
(12) Patent Application Publication
(10) Pub. No.: US 2008/0317867 A1
(43) Pub. Date: Dec. 25, 2008

(54) Cosmetic System, and Method for Disguising Skin Defects

(76) Inventor: Matthew A. Singer, Memphis, TN (US)

Correspondence Address: BAKER, DONELSON, BEARMAN, CALDWELL & BERKOWITZ, PC, 900 SOUTH GAY STREET, 2200 RIVERVIEW TOWER, KNOXVILLE, TN, 37902, US

(21) Appl. No.: 11/765,790

(22) Filed: Jun. 20, 2007

(51) Int. Cl. A61K 31/695; A61K 33/24; A61K 35/00; A61K 35/36; A61Q 1/02

(52) U.S. Cl. 424/543; 424/649; 514/63; 514/789

(57) ABSTRACT

The present invention provides a cosmetic system for disguising dermatological blemishes. The cosmetic system includes a viscous silicone material and a catalyst. The silicone material is pigmented with a cosmetic pigment according to a selected skin tone. In one aspect, the cosmetic system also includes a flocking agent. The flocking agent is mixed within the silicone material or the catalyst to provide texture. The cosmetic system also includes at least two cartridges. The cartridges hold the viscous silicone material and the catalyst, separately. Optionally, the cosmetic system includes a dispensing gun. The dispensing gun has a tip through which the silicone material and the catalyst may be dispensed, whereupon the silicone material and the catalyst are mixed and applied to a user's skin for curing. A method for disguising dermatological blemishes is also provided herein.

### FIELD OF THE INVENTION

The present invention relates to cosmetic systems. More specifically, the invention relates to a cosmetic system that may be used to obscure dermatological defects or to disguise skin blemishes.

### BACKGROUND OF THE INVENTION

Some persons carry with them a skin defect that may cause embarrassment. The defect may be a result of a surgical procedure, leaving permanent scarring. Alternatively, the defect may be a result of trauma such as an automobile accident or a fall. Alternatively, the defect may be significant skin blemishes due to acne scarring. In any of these instances, the person may be self-conscious about their appearance and seek to disguise or camouflage the skin defect.

Cosmetic materials are well-known as a means of enhancing or otherwise altering a person's appearance. Cosmetics are used to cover inconsistent skin pigmentation and wrinkles. However, standard cosmetics are not always suitable where the skin defect is profound.

For significant physiological defects, prostheses such as maxillofacial prosthetics are known. Such prosthetics may be of particular use for patients who have undergone the removal of a highly visible portion of their anatomy such as an ear, the nose, or an ocular orbit due to cancer. U.S. Pat. No. 4,623,593 discusses such a prosthetic. The '593 patent is entitled “Self-Adhesive Polymer Composition for Use as Prosthetic Appliance.” This discloses the use of polymeric materials for maxillofacial prosthetics.

The '593 patent notes that “[a] number of different types of polymeric materials have been utilized as base materials for prostheses.” The patent mentions “silicone rubbers and polyurethanes.” The '593 patent describes a molding process for the prosthetic that results in a tacky characteristic. The patent specifically states: “We have found that an integral surface layer having pressure-sensitive adhesive properties may be formed on an otherwise fully cured polymeric body. This may be accomplished by applying a sufficient amount of a cross-linking inhibition agent to selected surfaces of a mold cavity prior to packing the cavity with uncured monomeric, prepolymeric, or unvulcanized polymeric material. The mold is then closed and the material cured. The cross-linking inhibition agent acts on the surface or surfaces of the polymer to prevent complete cross-linking thereof. The body of the polymer is otherwise completely cured and has the same properties as would be expected.” The end product is offered in a cured state.

Maxillofacial prosthetics may be too expensive or too cumbersome in the case of “mere” skin defects. Therefore, a need exists for a cosmetic material that is able to disguise a profound skin defect such as surgical scarring or acne pitting. Further, a need exists for a cosmetic system that may be purchased by a consumer and applied to the skin defect to provide a translucent, realistic artificial skin.

U.S. Pat. No. 5,147,401 and U.S. Pat. No. 6,383,220 are each entitled “Artificial Skin.” These patents teach skin coverings to aid in the protection and healing of flesh wounds. There is no discussion concerning the temporary covering of skin defects or scars, nor is there a teaching of applying an uncured or uncatalyzed silicone-based material to the patient's skin.

### SUMMARY OF THE INVENTION

The present invention provides a cosmetic system for disguising dermatological blemishes. The cosmetic system includes a viscous silicone material and a catalyst. The cosmetic system also includes a pigment designed to tint the silicone material according to a selected skin tone. The pigment may be pre-mixed into either the silicone material, the catalyst, or both. The pigment may be a pigmented silicone oil, a pigmented cosmetic cream, or a pigmented powder. In one aspect, the cosmetic system also includes a flocking agent. The flocking agent is also mixed with either the silicone material, the catalyst, or both. The flocking agent serves to provide texture and, optionally, color once the silicone material has catalyzed on a user's skin.

The cosmetic system also includes at least two containers. The containers hold the viscous silicone material and the catalyst, separately. The containers may be jars. Alternatively, the containers may be cartridges configured to be loaded into a dispensing gun.

Optionally, the cosmetic system includes a dispensing gun. The dispensing gun has a tip through which the silicone material and the catalyst may be dispensed. Preferably, the silicone material and the catalyst are dispensed at substantially a 1:1 ration by volume. As the viscous components are extruded through the tip, the silicone material and the catalyst are mixed for curing.

A method for disguising dermatological blemishes is also provided herein. In one aspect, the method includes providing a viscous silicone material and a catalyst. Either the silicone material or the catalyst (or both) is pigmented with a cosmetic pigment according to a desired skin tone. In one aspect, the cosmetic system also includes a flocking agent. The flocking agent is mixed within the silicone material to provide texture.

The method also includes dispensing the silicone material and the catalyst from a dispensing gun. The dispensing gun has a tip through which the silicone material and the catalyst may be dispensed, whereupon the silicone material and the catalyst are mixed for curing. The viscous mix of silicone material and catalyst is applied to a dermatological area of interest. The method then includes allowing the new silicone mixture to cure.

A method for providing a cosmetic system is also provided herein. In one aspect, the method includes placing a viscous silicone material in a first cartridge, and placing a catalyst in a second cartridge. The method further includes providing a cosmetic pigment. The cosmetic pigment is tinted according to a desired or selected skin tone. The cosmetic pigment is pre-mixed with either the silicone material, the catalyst, or both.

In one aspect, the cosmetic system also includes a flocking agent. The flocking agent is mixed within the silicone material or the catalyst to provide texture and, optionally, color.

The first and second cartridges are arranged and configured to be received within a dispensing gun. The dispensing gun has a tip through which the silicone material and the catalyst may be together dispensed as a viscous mixture. The mixture is applied to a dermatological area of interest for curing.

The method further includes providing a dispensing gun for receiving the first cartridge and the second cartridge. The dispensing gun has a tip through which the viscous silicone material and the catalyst may be injected, whereupon the silicone material and the catalyst are mixed. In one aspect, the first and second cartridges are integral. In one aspect, the first and second cartridges are sized to provide silicone material and catalyst that is dispensed at substantially a 1:1 ration by volume.

### BRIEF DESCRIPTION OF THE DRAWINGS

So that the manner in which the recited features of the present invention can be better understood, certain flow charts and/or drawings are appended hereto. It is to be noted, however, that the appended artwork illustrates only selected embodiments of the inventions and are therefore not to be considered limiting of scope, for the inventions may admit to other equally effective embodiments and applications.

FIG. 1 is a perspective view of a dispensing gun as may be used as part of the cosmetic system of the present invention, in one embodiment.

FIG. 2 is a top, cross-sectional view of a portion of the dispensing gun of FIG. 1.

FIG. 3 is a perspective view of two cartridges for holding viscous liquids such as a silicone-based material and a catalyst.

FIG. 4 is a perspective view of a skin-texturing pad as may be used in connection with the cosmetic system, in one embodiment.

FIG. 5 is a perspective view of a skin-texturing pad in an alternate embodiment. The skin pad of FIG. 5 has finer features, and is designed to be used on the facial or neck areas of a patient.

### DESCRIPTION OF CERTAIN EMBODIMENTS

### Definitions

As used herein, the term “flocking agent” refers to a collection of tiny spikes or hairs. The flocking agent may be fabricated from, for example, short natural or synthetic hair or fibers. The short hairs are preferably colored.

The term “silicone material” refers to any viscous composition having a polymerized siloxane, or polysiloxane. Polysiloxanes have a silicon-oxygen backbone (Si—O—Si—O—Si—O . . . ) and an organic group (such as methyl, ethyl or phenyl) attached. The silicone material may be, for example, a linear silicone oil of the non-crosslinked polydimethylsiloxane (PDMS) type. Alternatively, the silicone material may be of the crosslinked organosiloxane type. The silicone material may be in the form of an emulsion. Suitable emulsions include but are not limited to water-in-silicone emulsions, oil-in water emulsions, and silicone-in-water emulsions.

The term “catalyst” refers to a material which, when combined with silicone, cures to form a substantially solid material. A non-limiting example is a platinum-type catalyst.

The term “dispensing gun” refers to any apparatus that dispenses a silicone material and a catalyst in such a manner that the silicone material and the catalyst are mixed for curing.

### DESCRIPTION OF SELECTED SPECIFIC EMBODIMENTS

The present invention provides a cosmetic system for disguising dermatological defects and blemishes. The cosmetic system is a silicone-based system that can be used to temporarily fill acne blemishes. Alternatively, the cosmetic system may be used to disguise more significant scars that may arise from surgeries, diseases or traumatic accidents.

The cosmetic system first comprises a silicone material. In one embodiment, the silicone material is fabricated from a dimethyl silicone such as polydimethylsiloxane. Such silicones are available, for example, from NuSil Technology of Carpinteria, Calif. However, the cosmetic systems herein are not limited to any particular type or source of silicone material, so long as the silicone material may be catalyzed at room temperature. Preferably, the silicone material has the viscosity of a gel or soft solid at room temperature conditions, prior to catalysis.

It is necessary for the silicone material to be cured, such as through a process of catalysis. Therefore, the cosmetic system next includes a catalyst. The cosmetic systems herein are not any limited to any particular type or source of catalyst. However, it is preferred that a non-toxic reaction occur during catalysis. A preferred catalyst is a platinum metal catalyst.

One particularly desired combination of silicone material and catalyst is the NuSil™ MED-6820. MED-6820 comes in Parts A and B, with Part A being the catalyst, and Part B being the silicone material. For catalysis, Parts A and B of MED-6820 may be degassed, and mixed at 50%/50% proportions. A 50 centistoke oil may be added to Part A, Part B, or both, prior to mixing. The oil serves as a diluent to facilitate extrusion through a dispenser (described below). Stated another way, the diluent reduces the viscosity of the silicone material. At higher percentages, the diluent also softens the final cured product, making it more pliable. This is of benefit for larger patch applications on a user's skin.

MED-6820 provides an advantageous combination of silicone material and catalyst. Parts A and B each provide high viscosity and temperature tolerance before catalysis, and good optical clarity upon mixing and curing. It should be added that a small amount of platinum accelerator may optionally be added to the catalyst to accelerate the curing or vulcanization process. Accelerators may reduce the life of a resulting silicone product. However, the present application is designed to be temporary, i.e., lasting less than 24 hours before it is removed.

As noted, the silicone material and the catalyst are mixed to provide a silicone mixture. In one aspect, the silicone mixture is custom-blended. This means that the mixture includes a cosmetic pigment that has been added to match a selected skin tone. Using pigments, various tints or shades of silicone mixture may be provided so that customers may select an intrinsically colored product that most closely matches their own skin tone.

The pigment is preferably a cosmetic pigment that is fabricated from a pressed cream. Examples of a suitable pressed cream include cream-based makeup. The cream may be water-based, silicone-based or petroleum-based.

As an alternative, the pigment may be a powder such as a cosmetic make-up powder. Alternatively, the pigment may be a silicone pigment. Various tones or colors of silicone pigment are available from NuSil Technology. Also, Eager Plastics, Inc. of Chicago, Ill. provides opaque silicone pigments having no/low odor and toxicity.

The pigment may be pre-mixed with the silicone material. Alternatively, the pigment may be pre-mixed with the catalyst, though preferably the pigment would not be a silicone-based cream in this instance. Alternatively still, the pigment may be supplied as a separate component and mixed with both the silicone material and the catalyst at the time the mixture is formed by a user. In this latter instance, the pigment will need a carrier material such as a low-viscosity silicone compound.

The cosmetic system optionally also includes a flocking agent. The flocking agent is used to provide depth and texture to the cosmetic mixture. The flocking agent may also provide color. Preferably, the flocking agent is pre-mixed with the silicone material. However, it may alternatively be pre-mixed with the catalyst. Alternatively still, the flocking agent may be supplied as a separate component with a pigment, and then mixed with both the silicone material and the catalyst at the time the mixture is formed by a user.

The flocking agent is preferably natural hair cut in very short sections, such as 5 to 25 mm. However, any material that is capable of providing texture to the silicone material may be used. Non-limiting examples include natural fibers, e.g., cotton and pulp, or synthetic fibers. The flocking agent may be of any color. However, in one aspect the flocking agent has a brown or black color so that flocking agent may be added to the silicone material to selectively darken the final mixture.

In order to dispense the silicone material onto a patient's skin, a dispensing gun is preferably employed. Thus, in one embodiment the cosmetic system includes a dispensing gun. The dispensing gun is preferably a dual chamber applicator that receives two cartridges. A first cartridge holds the silicone material, while a second cartridge holds the catalyst.

In one aspect, the dispensing gun is the dispenser that was the subject of U.S. Pat. No. 5,333,760, entitled “Dispensing and Mixing Apparatus.” The assignee of the '760 patent is listed as Coltene/Whaledent, Inc. of Mahwah, N.J. This patent is incorporated herein to the extent it is consistent with the present disclosure.

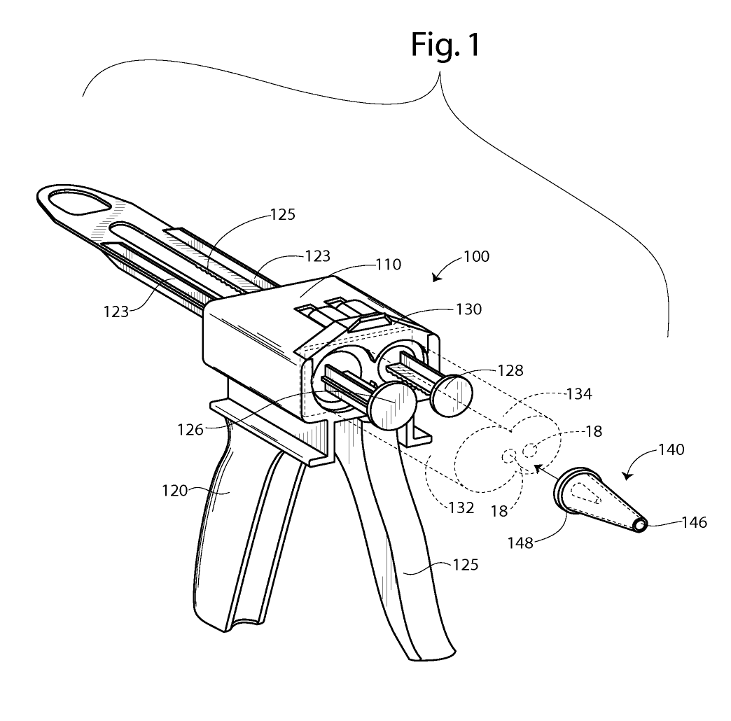

FIG. 1 provides a schematic view of another dispensing gun 100 as may be used in the cosmetic system herein. The illustrative gun 100 is a dual cartridge dispensing apparatus for dispensing and mixing the viscous components of the cosmetic system, to wit, the silicone material and the catalyst. The silicone material and the catalyst are held in respective cartridges. The cartridges are seen in phantom at 132 and 134, indicating that they are not part of the dispensing gun 100 itself. The first cartridge 132 is pre-packed with the silicone material, while the second cartridge 134 is pre-packed with the catalyst. Preferably, the silicone material in the first cartridge 132 is pre-mixed with the pigment and the flocking agent.

As seen in FIG. 1, the dispensing gun 100 includes a body 110. The body 110 supports other components of the dispensing gun 100 as described below. Operatively connected to the body 110 is ratcheting system. The ratcheting system comprises a handle 120 and an actuating trigger 125. By repetitively squeezing the trigger 125, the silicone material and the catalyst are urged under pressure from the respective cartridges 132, 134.

The dispensing gun 100 also includes a chamber 130. The chamber 130 may be an elongated receptacle for receiving the cartridges, as in the dispensing apparatus of the '760 patent. In the '760 patent, the chamber is separated into two compartments, with each of the compartments receiving a separate cartridge in which the viscous components are stored. However, in the arrangement of FIG. 1 the chamber defines a slot. The slot 130 receives a plate (seen at 16 in FIG. 3) that in turn connects to and supports the two cartridges 132, 134.

The dispensing gun 100 also includes a pair of pistons 126, 128. The pistons 126, 128 act against the viscous materials in the cartridges 132, 134 in order to urge the materials from the cartridges 132, 134. Each of the pistons 126, 128 is carried at the end of a respective rail 123. The rails 123 travel forward through the body 110 and the chamber 130 of the dispensing gun 100 as the actuating trigger 125 is repetitively squeezed. The rails 123 include a toothed edge 125 which advance the pistons 126, 128 as part of the ratcheting system.

It is understood that the ratcheting system further includes gears, teeth or other advancing mechanism within the body. This commonly known feature is not illustrated or further described herein.

The dispensing gun 100 next includes a mixing tip 140. The use of a mixing tip, or “static mixer,” is known in the art of mixing extruded components. The mixing tip may be of various types such as those described in U.S. Pat. Nos. 4,538,920; 3,923,288; 3,635,444, or others. In one arrangement, the mixing tip is integral to the chamber. This type of arrangement is shown in the dispensing apparatus of the '760 patent.

In the illustrative arrangement of FIG. 1, the mixing tip 140 defines a separate member. This means that the mixing tip 140 may be disconnected from other components for cleaning or even replacement. The mixing tip 140 includes a central bore 145 through which the viscous silicone material and the catalyst flow from the cartridges 132, 134 when the ratcheting system is actuated.

The mixing tip 140 of FIG. 1 includes a flanged proximate end 148. The flanged proximate end 148 is configured to snap into a bracket formed on the cartridges 132, 134. (The bracket is seen at 22 in FIG. 3 at the distal end 12 of the cartridges 132, 134.) In this way, a sealed connection between the cartridges 132, 134 and the mixing tip 140 may be acquired.

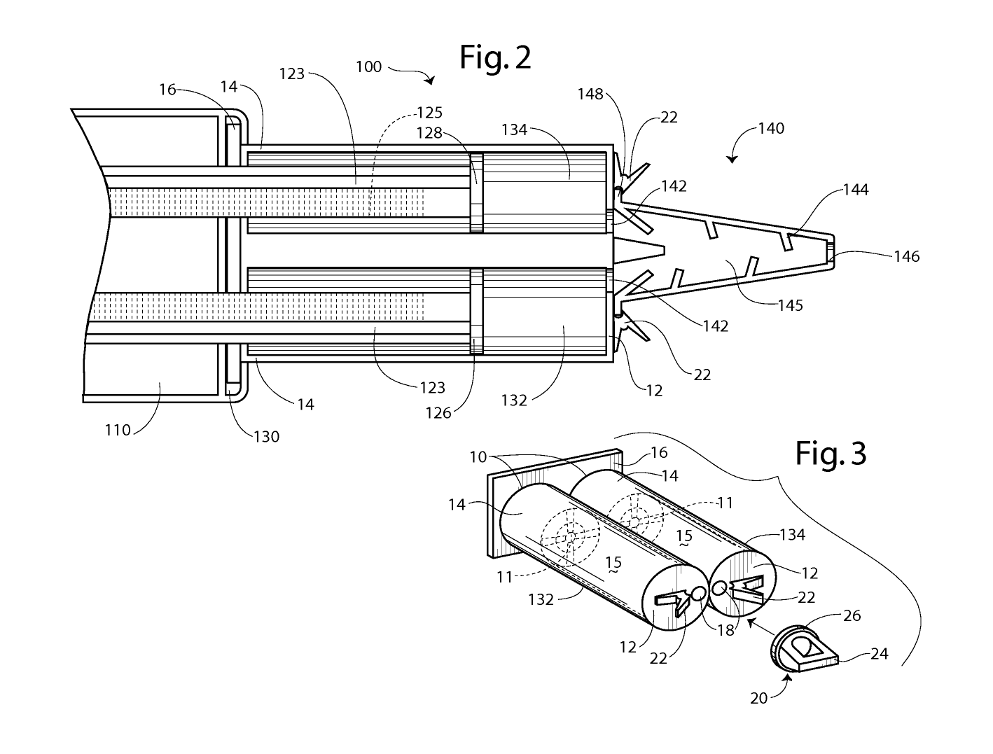

FIG. 2 is a top, cross-sectional view of a portion of the dispensing gun 100 of FIG. 1. In this view, certain features of the dispensing gun 100 are more clearly seen. The cartridges 132, 134 have been removed so that the rails 125 are visible. As noted, the rails advance through the body 110 in response to ratcheting of the trigger 125. Also visible is the slot 130. Again, the slot 130 receives the plate 16 that supports the cartridges 132, 134.

Also seen in FIG. 1 is the mixing tip 140. The mixing tip 140 is shown at the end of the rails 125. However, it is understood that the mixing tip 140 is actually connected to the distal ends 12 of the respective cartridges 132, 134, as described above.

As noted, the mixing tip 140 defines a central bore 145. The central bore 145 receives the viscous silicone material and the catalyst as they are urged from the dispensing gun 100. The viscous components are mixed as they travel through the bore 145. Preferably, the central bore 145 has a plurality of stationary mixing vanes 144 internally of the bore 145. The mixing vanes 144 can be of various shapes so long as they assist the components in being mixed together as they are forced through the bore 145.

Openings 142 are provided at the proximate flanged end 148 of the mixing tip 140. The openings 142 receive the viscous components as they are urged from the cartridges 132, 134. The components travel through the bore 145 and exit the mixing tip through a distal end 146.

The dispensing gun 100 shown and described in connection with FIGS. 1 and 2 is merely illustrative. It is understood that any dispensing apparatus capable of moving the viscous components of the cosmetic system from the cartridges such that they are mixed and catalyzed may be employed.

FIG. 3 is a perspective view of two cartridges 132, 134 for holding the viscous components, to wit, the silicone-based material and the catalyst. The two cartridges 132, 134 are sized to be received within the chamber 130 of the gun 100. In the illustrative arrangement, the plate 16 slides into the slot that defines the chamber 130. The cartridges 132, 134 are preferably substantially circular in cross-section. Preferably, the cartridges 132, 134 are fabricated from a transparent or translucent plastic material.

Each cartridge 132, 134 defines an inner bore 15 for receiving a viscous material. The diameter of the inner bore 15 may range from about 1.0 to about 3.0 cm, although this range is simply illustrative. The cartridges 132, 134 may have different inner diameters, or equivalent inner diameters. Each cartridge 132, 134 has a posterior end 10 and an anterior end 12. The posterior ends 10 each receive a sealed cap 11. The sealed cap 11 is movable along the inner bore 15 of the cartridges 132, 134 in response to force applied by the pistons 126, 128. The anterior ends 12 of the cartridges 132, 134 include an outlet 18. The outlets 18 in the cartridges 132, 134 are aligned with the openings 142 in the mixing tip 140.

The cartridges 132, 134 shown in FIG. 3 are integral. In this respect, the posterior ends 14 of the cartridges 132, 134 are connected to a plate 16. The plate 16, in turn, slides into the chamber 130 in the dispensing gun 100. The anterior ends 12 of the cartridges 132, 134 form a bracket 22. The bracket 22, in turn, receives a cap 20. The cap 20 slides into the opposing halves making up the bracket 22 in order to provide a seal to the openings 18 before the cartridges 132, 134 are loaded into the dispensing gun 100. The cap 20 includes a small handle 24 for gripping the cap 20.

When the dispensing gun 100 is ready for use, the cap 20 is removed from the cartridges 132, 134. The cartridges 132, 134 are then inserted into the slot defining the chamber 130. The mixing tip 140 is then slid into the bracket 22. In one aspect, the mixing tip 140 is rotated in order to lock the mixing tip 140 onto the bracket 22.

It is noted that the two cartridges 132, 134 may be separate items, that is, not permanently connected. It is also noted that more than two cartridges might be used in dispensing viscous fluids in the cosmetic system. For instance, a first cartridge might hold a silicone material, a second cartridge might hold a catalyst, and a third cartridge might hold a pigment in a carrier material. In this instance, the third cartridge would have a much smaller bore than the first and second cartridges. The flocking material may be included in any of the three cartridges or even all three.

The cartridges 132, 134 each contain a viscous component. The viscous components are extruded from the cartridges 132, 134 and through the openings 18 in response to action of the dispensing trigger 125. More specifically, the trigger 125 urges the pistons 126, 128, which in turn act against the sealed caps 11 at the posterior ends 10 of the cartridges 132, 134. Ultimately, the viscous components are extruded into the mixing tip 140 and out the distal end 146. In this way, the silicone material and the catalyst are mixed to form a silicone mixture so that a cosmetic material may be cured on the skin surface of a user. In one aspect, the cured silicone mixture has a durometer of about 5 to 25.

The cosmetic system may also comprise a skin prepping solution. The skin prepping solution is designed to remove oil and dirt from a dermatological area of interest. For example, the skin prepping solution may be ethyl acetate. The skin prepping solution is applied to the skin before the silicone material is dispensed.

After prepping, the silicone mixture may be applied to the patient's skin in different ways. First, the mixture may be applied directly to the skin as the mixture is extruded from the tip 140 of the dispenser 100. Alternatively, the mixture may be squeezed onto a dish (not shown) and then applied to the skin by use of a spatula or other tool. Alternatively still, the mixture may be squeezed into the user's hand or into a dish, and then applied to the area of concern by hand. In any instance, the silicone mixture takes about 5 to 10 minutes to cure. Upon curing, the mixture constitutes a solid synthetic elastomer.

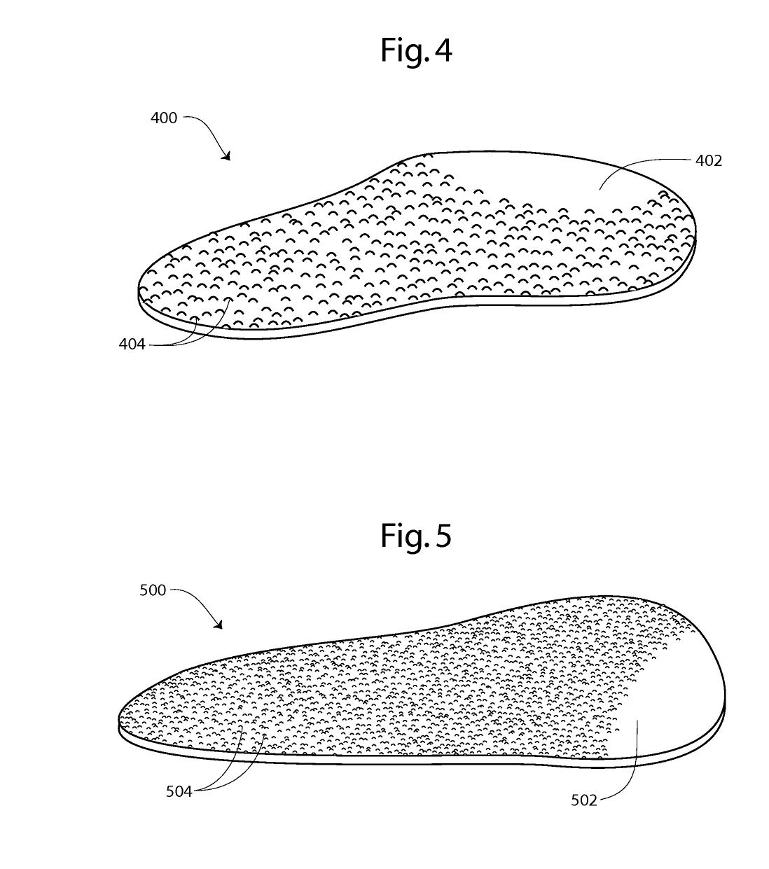

The cosmetic system may also comprise a skin texturing pad. FIG. 4 presents an illustrative skin texturing pad 400. The skin texturing pad 400 defines a thin, flexible substrate 402 having a plurality of dimples 404. In the arrangement 400, the dimples are large, and may be used to provide texture to a user's hand, arm, legs or foot, for example. Thus, the pad 400 has a texture designed to match the porous pattern of the dermatological area of interest.

A separate skin texturing pad may be used on dermatological areas having finer features. FIG. 5 presents such a skin texturing pad 500. The skin texturing pad 500 again defines a thin, flexible substrate 502 having a plurality of dimples 504. However, in the arrangement 500, the dimples are more subtle, and may be used to provide texture to a user's face, neck, or stomach, for example. In either instance, the use of a skin texturing pad 400 or 500 tends to reduce the shine or light reflection from the cured silicone material.

The pads 400, 500 are preferably fabricated from urethane. Pads may optionally be acquired from Smooth-On, Inc. of Easton, Pa. After the pad 400 or 500 has been applied to the silicone, it is carefully removed. A matte skin cover disguising a dermatological blemish is left. The final material is dry to the touch, transparent, water resistant, durable, flexible, and has a natural texture. Under normal conditions it is anticipated that the dried material will last up to 16 hours.

A method is provided herein for disguising dermatological blemishes. In one aspect, the method includes providing a viscous silicone material and a catalyst. The silicone material or the catalyst (or both) is pigmented with a pigment according to a desired skin tone. In one aspect, the cosmetic system also includes a flocking agent. The flocking agent is pre-mixed with the silicone material or the catalyst (or both) to provide texture.

The method also includes dispensing the silicone material and the catalyst from a dispensing gun. The dispensing gun has a tip through which the silicone material and the catalyst may be dispensed, whereupon the silicone material and the catalyst are mixed for curing. The viscous mixture of silicone material and catalyst is applied to a dermatological area of interest. The method then includes allowing the silicone material to cure.

In one embodiment, the method also includes the step of providing a skin prepping solution to the dermatological area of interest. The skin prepping solution is applied prior to applying the silicone material to a dermatological area of interest. In one aspect, the skin preparation creates a thin film that serves as a barrier to prevent the silicone material from sticking too firmly to the skin. In another aspect, the prepping material helps to remove any oils or dirt that may be present on the skin which may interfere with adhesion.

A method for providing a cosmetic system is also provided herein. In one aspect, the method includes placing a viscous silicone material in a first cartridge. The silicone material is pigmented with a cosmetic pigment according to a selected skin tone. In one aspect, the cosmetic system also includes a flocking agent. The flocking agent is mixed within the silicone material to provide texture.

The method also includes placing a catalyst in a second cartridge. The first and second cartridges are arranged and configured to be received within a dispensing gun. The dispensing gun has a tip through which the silicone material and the catalyst may be together dispensed as a viscous silicone mixture. The mixture is applied to a dermatological area of interest for curing.

The method further includes providing a dispensing gun for receiving the first cartridge and the second cartridge. The dispensing gun has a tip through which the viscous silicone material and the catalyst may be injected, whereupon the silicone material and the catalyst are mixed.

Optionally, the method also includes applying a skin texturing pad over the silicone material. The pad is texturized in accordance with the dermatological area of interest, and provides depth and/or texture. The skin texturizing pad is removed before the silicone material fully cures.

In one aspect, the method further comprises applying facial makeup to the dermatological area of interest. The facial makeup is applied prior to applying the silicone material to the dermatological area of interest. It is noted that the silicone material dries translucently, and additional makeup need not be placed over the silicone material. The custom blend of the silicone material, the catalyst and the pigment is preferably designed so that an element of translucence is maintained. Ideally, the cured silicone mixture should not interfere with the user's skin appearance, but should blend in so as not to be notice. However, in some instances a user may have a scar that has left a different skin coloration than their natural skin. In this instance, the user may choose to use a special makeup that blends in the color of the scar tissue with the surrounding skin.

The first and second cartridges are preferably integral to one another. In one embodiment, the method includes selling the first and second cartridges together to be inserted into the dispensing gun. The patient selects the “kit” that most closely matches his or her skin tone.

Ancillary to the cosmetic system is a consideration as to pigmentation for users in the market place. The human race presents a true “kaleidoscope” of colors. In one aspect, it is desirable to select a minimum number of pre-mixed cartridges which nevertheless support the “skin kaleidoscope” presented by the marketplace. In another aspect, it is desirable to offer a color-matching system wherein a silicone material is custom blended specifically for an individual user by using a computer-driven color matching system.

The foregoing description and examples have been set forth merely to illustrate the inventions herein and are not intended to be limiting. Since modifications of the disclosed embodiments incorporating the spirit and substance of the inventions may occur to persons skilled in the art after reading this disclosure, the inventions should be construed broadly to include all variations falling within the scope of the appended claims and equivalents thereof.

For example, it is within the spirit and scope of the present invention to dispense and mix the silicone material and the catalyst manually. The silicone material and the catalyst may be provided as part of a cosmetic system in separate jars or other containers. The jars may be sold, for example, in a bundled manner, or sold separately. In any event, the silicone material and the catalyst are preferably mixed at substantially a 1:1 ratio by volume in order to form the silicone mixture.

It is also noted that the cosmetic system may be sold without a dispensing gun. In one aspect, the silicone material and the catalyst are sold in cartridges, which are then loaded into the dispensing gun that is separately sold or acquired. In another aspect, the silicone material and the catalyst are sold in jars. The silicone material and the catalyst may be manually placed into empty cartridges, which are then loaded into the dispensing gun. Thus, various arrangements and embodiments for the cosmetic system and the method for disguising skin defects are contemplated herein.

What is claimed is:

1. A cosmetic system for disguising dermatological blemishes, comprising:
a viscous silicone material;
a pigment designed to reflect a skin tone;
a flocking agent to provide texture;
a catalyst; and
containers for separately holding the silicone material and the catalyst for mixing and dispensing onto a user's skin for curing.

2. The cosmetic system of claim 1, wherein the pigment is dispersed in the silicone material, in the catalyst, or both.

3. The cosmetic system of claim 1, wherein the flocking agent is dispersed in the silicone material, in the catalyst, or both.

4. The cosmetic system of claim 1, further comprising:
a dispensing gun having a tip through which the silicone material and the catalyst may be dispensed as a mix for application and curing; and
wherein the containers define cartridges configured to be received in the dispensing gun.

5. The cosmetic system of claim 4, wherein the cartridges are integral to one another.

6. The cosmetic system of claim 4, wherein the cartridges are sized such that the silicone material and the catalyst are dispensed from the dispensing gun in substantially a 1:1 volume ratio.

7. The cosmetic system of claim 1, wherein the containers define separate jars.

8. The cosmetic system of claim 1, further comprising:
a skin prepping solution.

9. The cosmetic system of claim 4, wherein the skin prepping solution comprises ethyl acetate.

10. The cosmetic system of claim 1, further comprising:
at least one skin texturizing pad.

11. The cosmetic system of claim 1, wherein the silicone material comprises a platinum, RTV silicone material.

12. The cosmetic system of claim 1, wherein the silicone material comprises Part B of MED-6820 from Nusil Technology.

13. The cosmetic system of claim 1, wherein the flocking agent comprises natural hair, natural fibers, or synthetic fibers.

14. The cosmetic system of claim 1, wherein the catalyst comprises a platinum metal catalyst.

15. The cosmetic system of claim 1, wherein the silicone material and the catalyst, when mixed, form a translucent material having a durometer of about 5 to 25.

16. The cosmetic system of claim 1, wherein the pigment is a cosmetic pigment fabricated from a pressed cream or a powder.

17. The cosmetic system of claim 1, wherein the silicone material further comprises a 50 centistoke oil.

18. The cosmetic system of claim 1, wherein the catalyst further comprises an accelerator.

19. A method for disguising dermatological blemishes, comprising:
providing a viscous silicone material;
providing a catalyst;
dispensing the silicone material and the catalyst from a dispensing gun, thereby mixing the silicone material and the catalyst into a silicone mixture for application and curing;
applying the silicone mixture to a dermatological area of interest; and
allowing the silicone material to cure.

20. The method of claim 19, wherein the pigment is dispersed in the silicone material, in the catalyst, or both.

21. The method of claim 19, wherein the flocking agent is dispersed in the silicone material, in the catalyst, or both.

22. The cosmetic system of claim 19, further comprising:
providing a cartridge for containing the silicone material; and
providing a cartridge for containing the catalyst;
the cartridges being configured to be received in the dispensing gun.

23. The cosmetic system of claim 22, wherein the cartridges are integral to one another.

24. The cosmetic system of claim 22, wherein the cartridges are sized such that the silicone material and the catalyst are dispensed from the dispensing gun in substantially a 1:1 volume ratio.

25. The method of claim 19, further comprising:
providing a skin prepping solution to the dermatological area of interest prior to applying the silicone mixture to a dermatological area of interest.

26. The method of claim 19, further comprising:
applying a skin texturizing pad over the silicone mixture, the skin texturizing pad being textured in accordance with the dermatological area of interest; and
removing the skin texturizing pad before the silicone mixture fully cures.

27. The method of claim 19, further comprising:
applying facial makeup to the dermatological area of interest prior to applying the silicone mixture to the dermatological area of interest.

28. A method for providing a cosmetic system, comprising:
placing a silicone material in a first cartridge, the silicone material being pigmented according to a patient's skin tone;
placing a catalyst in a second cartridge, the first and second cartridges being arranged and configured to be received within a dispensing gun, the dispensing gun having a tip through which the silicone material and the catalyst may be together dispensed as a silicone mixture for curing;
providing a dispensing gun for receiving the first cartridge and the second cartridge, the dispensing gun having a tip through which the silicone material and the catalyst may be injected, whereupon the silicone material and the catalyst are mixed at a substantially 1″1 ration by volume into the silicone mixture.

29. The method of claim 28, further comprising:
providing a skin prepping solution to create adhesion to a dermatological area of interest, and to insulate skin at the dermatological area of interest.

30. The method of claim 28, further comprising:
providing a skin texturing pad for imbuing texture to the silicone mixture on a user's skin during curing.
